(19) United States
(12) Patent Application Publication
(10) Pub. No.: US 2018/0051051 A1
(43) Pub. Date: Feb. 22, 2018

(54) METHOD FOR PREPARING PRECURSOR OF GENE EXPRESSION PROBE

(71) Applicant: INSTITUTE OF NUCLEAR ENERGY RESEARCH, ATOMIC ENERGY COUNCIL, EXECUTIVE YUAN, R.O.C., TAOYUAN COUNTY (TW)

(72) Inventors: SHOW-WEN LIU, TAOYUAN COUNTY (TW); CHENG-FANG HSU, TAOYUAN COUNTY (TW); YU CHANG, TAOYUAN COUNTY (TW)

(21) Appl. No.: 15/242,798

(22) Filed: Aug. 22, 2016

(51) Int. Cl. C07H 23/00; C07B 59/00; A61K 51/04

(52) U.S. Cl. CPC C07H 23/00; C07B 2200/05; A61K 51/0491; C07B 59/005

(57) ABSTRACT

A method for preparing a precursor of gene expression probe is revealed. First prepare 2-deoxy-2-fluoro-3,5-di-O-benzoyl-α-D-arabinofuranosyl bromide and 2,4-bis-O-(trimethylsilyl)-5-bromouracil respectively. Then use 2-deoxy-2-fluoro-3,5-di-O-benzoyl-α-D-arabinofuranosyl bromide and 2,4-bis-O-(trimethylsilyl)-5-bromouracil to perform a coupling reaction and get a 1-(3,5-di-O-benzoyl-2-deoxy-2-fluoro-β-D-arabinofuranosyl)-5-bromouracil. Next use 1-(3,5-di-O-benzoyl-2-deoxy-2-fluoro-β-D-arabinofuranosyl)-5-bromouracil generated and hexabutylditin to perform a substitution reaction in an anhydrous 1,4-dioxane solution and get 1-(3,5-di-O-benzoyl-2-deoxy-2-fluoro-β-D-arabinofuranosyl)-5-tributylstannyl)uracil. At last carry out debenzoylation of 1-(3,5-di-O-benzoyl-2-deoxy-2-fluoro-β-D-arabinofuranosyl)-5-tributylstannyl)uracil to get 5-tributylstannyl-1-(2-deoxy-2-fluoro-β-D-arabinofuranosyl)uracil (FSAU), a precursor of gene expression probe.

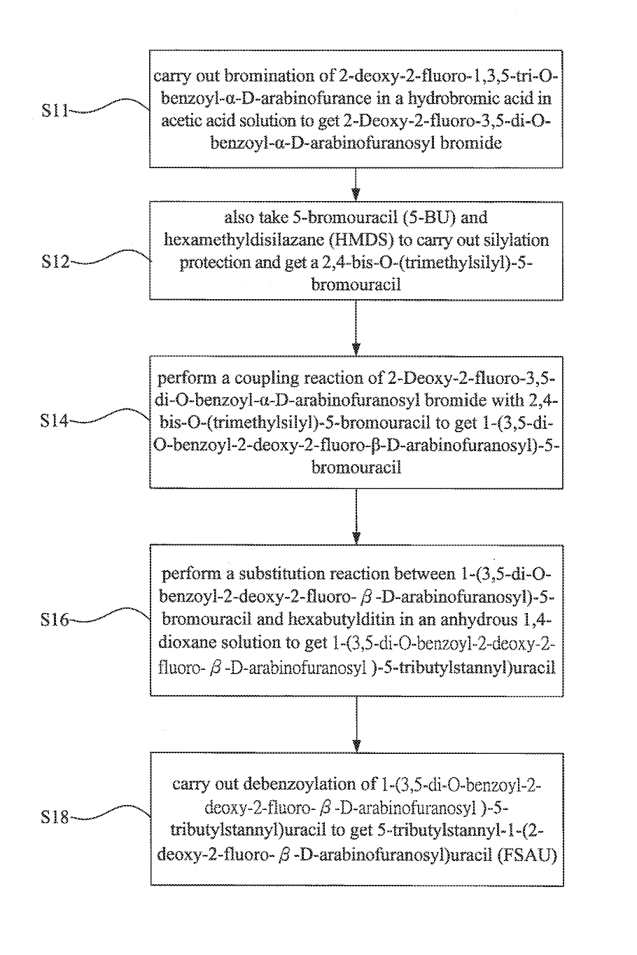

### BACKGROUND OF THE INVENTION

### Field of the Invention

The present invention relates to a method for preparing a compound, especially to a method for preparing a precursor of gene expression probe.

### Descriptions of Related Art

Gene therapy is the medical transfer of therapeutic genes/DNA into humans by vectors. The genes transferred into patients express a corresponding protein to treat or eliminate inherited diseases or physical conditions caused by genetic defects. Several key factors affect therapeutic effects of gene therapy including vectors and DNAs. The vector should be highly contagious for effective transfer of DNA to target cells without immune responses or side effects. The DNA delivered into patient's cells will not cause changes in genes or any others. The DNA transferred to human bodies can make functional proteins in target cells smoothly and effectively. How to check and confirm whether therapeutic genes/DNA has been transferred to the target cells successfully is also a significant factor.

Owing to difficulty in detecting expression of the therapeutic genes/DNA in cells, a reporter gene has been developed and applied. Generally, the reporter gene is designed to be transferred into the target cells along with the therapeutic genes/DNA. The reporter gene is not only used to confirm whether the therapeutic genes/DNA are cloned into lesions of the target cells but also used to check expression of the therapeutic genes/DNA indirectly. The reporter gene is easily detected and featuring on sensitive, quantitative, and reproducible properties. Now most of reporter genes are detected by molecular imaging. That means molecular or cellular changes in vivo are observed by optical imaging, magnetic resonance imaging (MRI), microbubbles for contrast ultrasound imaging or Gamma-ray imaging. Moreover, the expression of the reporter gene is monitored by promoter expression, enhancer expression, gene expression, and the transfection system after the target gene being transferred into the cell so as to determine expression of the therapeutic gene/DNA in the target cell.

Herpes Simplex Virus 1 thymidine kinase reporter gene, HSV1-tk is a commonly used reporter gene now. Together with the therapeutic gene/DNA, the HSV1-tk is delivered into the target cell by the vector. The HSV1-tk gene is activated by constitutive or inductive promoter and transcribed to form messenger RNA (mRNA). Then the mRNA is translated to build HSV1-TK protein that works in the cell. In order to learn expression of HSV1-TK protein, radiolabeled nucleoside analogues such as fialuridine (FIAU) that passes and enters the cell membrane freely is used. HSV1-TK protein can phosphorylate radiolabeled nucleoside analogues in the cell so that the radiolabeled nucleoside analogues are unable to freely pass through the cell membrane freely and retained intracellularly. At the moment, the amount of radioactive substances accumulated in the cell directly signals HSV 1-TK activity and HSV1-tk gene expression. The expression of the therapeutic gene delivered into the cell together with HSV1-tk can further be determined.

The nucleoside analogues mentioned above are obtained by replacement of a 5-carbon sugar a part of nitrogenous base structure of purine nucleosids or pyrimidine nucleosides in human bodies. fialuridine (FIAU) is one of the common nucleoside analogues. A methyl group on the 5′ carbon position of the nitrogenous base is replaced by an iodine atom and a hydrogen atom on the 2′ carbon position of the 5-carbon sugar is replaced by a fluorine atom so as to get FIAU. The iodine atom can be further replaced by iodine-124 to form a radioactive tracer. The iodine-124 FIAU not only shows better imaging effect, its side effect on human bodies is also lowered. However, the expensive FIAU results in a high research cost. In order to reduce cost of gene-therapy related clinical research significantly, it's an important issue to develop a method for easily preparing a FIAU precursor with high yield rate and low production cost. Moreover, the precursor is easily and effectively reacted to form [¹²⁴I]FIAU that is further applied to related gene therapy.

### SUMMARY OF THE INVENTION

Therefore it is a primary object of the present invention to provide a method for preparing a precursor of gene expression probe. The precursor of gene expression probe produced includes a leaving group easy to be replaced. Thus the precursor reacts with iodine isotopes and the leaving group is replaced by the iodine isotopes easily. Therefore the gene expression probe is produced with higher production efficiency.

It is another object of the present invention to provide a method for preparing a precursor of gene expression probe that simplifies the steps required, shortens production time, reduces production cost and improves production efficiency significantly.

It is a further object of the present invention to provide a method for preparing a precursor of gene expression probe in which reactions of intermediate steps continue without any complicated separation and purification processes. Thus not only production time is saved, the yield rate of intermediate products can also be increased significantly. Thus more products are obtained with lower cost. The cost is reduced and the production efficiency is increased.

In order to achieve the above objects, a method for preparing a precursor of gene expression probe according to the present invention includes a plurality of steps. Firstly carry out bromination of 2-deoxy-2-fluoro-1,3,5-tri-O-benzoyl-α-D-arabinofurance in a hydrobromic acid in acetic acid solution to get 2-deoxy-2-fluoro-3,5-di-O-benzoyl-α-D-arabinofuranosyl bromide. Also take 5-bromouracil (5-BU) and hexamethyldisilazane (HMDS) to carry out silylation protection and get 2,4-bis-O-(trimethylsilyl)-5-bromouracil. Then perform a coupling reaction of 2-deoxy-2-fluoro-3,5-di-O-benzoyl-α-D-arabinofuranosyl bromide with 2,4-bis-O-(trimethylsilyl)-5-bromouracil to get 1-(3,5-di-O-benzoyl-2-deoxy-2-fluoro-β-D-arabinofuranosyl)-5-bromouracil. Next perform a substitution reaction between 1-(3,5-di-O-benzoyl-2-deoxy-2-fluoro-β-D-arabinofuranosyl)-5-bromouracil generated and hexabutylditin in an anhydrous 1,4-dioxane solution to get 1-(3,5-di-O-benzoyl-2-deoxy-2-fluoro-β-D-arabinofuranosyl)-5-tributylstannyl)uracil. At last, carry out debenzoylation of 1-(3,5-di-O-benzoyl-2-deoxy-2-fluoro-β-D-arabinofuranosyl)-5-tributylstannyl)uracil to get 5-tributylstannyl-1-(2-deoxy-2-fluoro-β-D-arabinofuranosyl)-uracil (FSAU). The preparation of the precursor of gene expression probe has been completed.

The weight percent of the hydrobromic acid in acetic acid solution used for bromination is ranging from 20% to 40%.

The reaction temperature of the bromination is ranging from −196° C. to 13° C.

The reaction time of the bromination is ranging from 15 hours to 25 hours.

An ammonium sulfate solution and hexamethyldisilazane are used for silylation protection.

The reaction temperature of the silylation protection is ranging from 120° C. to 160° C.

The reaction time of the silylation protection is ranging from 2 hours to 6 hours.

The reaction temperature of the coupling reaction is ranging from 70° C. to 110° C.

The reaction time of the coupling reaction is ranging from 15 hours to 25 hours.

The catalyst used in the substitution reaction is bis(triphenylphosphine)palladium chloride.

The reaction temperature of the substitution reaction is ranging from 90° C. to 130° C.

The reaction time of the substitution reaction is ranging from 18 hours to 30 hours.

The debenzoylation takes place in an ammonium hydroxide solution whose weight percent concentration is ranging from 15% to 25%.

The reaction temperature of the debenzoylation is ranging from 20° C. to 30° C.

The reaction time of the debenzoylation is ranging from 18 hours to 30 hours.

After the debenzoylation, perform a substitution reaction of 5-tributylstannyl-1-(2-deoxy-2-fluoro-β-D-arabinofuranosyl)uracil (FSAU) with radioactive iodine to get fialuridine labeled with radioactive iodine (I).

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

The FIGURE is a flow chart showing steps for preparing a compound of an embodiment according to the present invention.

### DETAILED DESCRIPTION OF THE PREFERRED EMBODIMENT

In order to learn features and functions of the present invention, please refer to the following embodiments and related descriptions with details.

To overcome the high cost problem of fialuridine labeled with radioactive iodine (I) used in clinical research and treatment, the present invention provides a method for preparing a new compound that improves preparation of a precursor of fialuridine labeled with radioactive iodine (I), 5-tributylstannyl-1-(2-deoxy-2-fluoro-β-D-arabinofuranosyl) uracil (FSAU). The present method not only simplifies the production process of the precursor by using different materials and reaction steps, but also provides higher recovery rate of extraction for reducing a waste during the production process and increasing the yield rate. Thus the production cost is reduced dramatically.

The followings are detailed descriptions of the components, properties, and steps related to a method for preparing a precursor of gene expression probe of the present invention.

Refer to the FIGURE, is a flow chart showing steps of a method for preparing a precursor of a gene expression probe according to the present invention is revealed. The method includes the following steps:

Step S11: perform bromination of 2-deoxy-2-fluoro-1,3,5-tri-O-benzoyl-α-D-arabinofurance in a hydrobromic acid in acetic acid solution to get 2-deoxy-2-fluoro-3,5-di-O-benzoyl-α-D-arabinofuranosyl bromide;
Step S12: also take 5-bromouracil (5-BU) and hexamethyldisilazane (HMDS) to carry out silylation protection and get 2,4-bis-O-(trimethylsilyl)-5-bromouracil.
Step S14: perform a coupling reaction of 2-deoxy-2-fluoro-3,5-di-O-benzoyl-α-D-arabinofuranosyl bromide with 2,4-bis-O-(trimethylsilyl)-5-bromouracil to get 1-(3,5-di-O-benzoyl-2-deoxy-2-fluoro-β-D-arabinofuranosyl)-5-bromouracil;
Step S16: use the 1-(3,5-di-O-benzoyl-2-deoxy-2-fluoro-β-D-arabinofuranosyl)-5-bromouracil generated and hexabutylditin to perform a substitution reaction in an anhydrous 1,4-dioxane solution and get 1-(3,5-di-O-benzoyl-2-deoxy-2-fluoro-β-D-arabinofuranosyl)-5-tributyl-stannyl)uracil;
Step S18: carry out debenzoylation of 1-(3,5-di-O-benzoyl-2-deoxy-2-fluoro-β-D-arabinofuranosyl)-5-tributylstannyl)-uracil to get 5-tributylstannyl-1-(2-deoxy-2-fluoro-β-D-arabinofuranosyl)uracil (FSAU).

As shown in the step S11, the bromination of 2-deoxy-2-fluoro-1,3,5-tri-O-benzoyl-α-D-arabinofurance, as an initial reactant, in a hydrobromic acid in acetic acid solution that gets 2-deoxy-2-fluoro-3,5-di-O-benzoyl-α-D-arabinofuranosyl bromide is represented by the following reaction formula one:

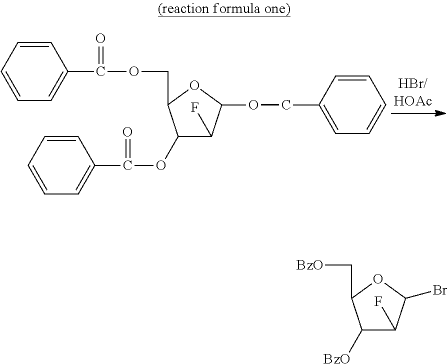

In this step, bromination of a benzoyl group of the 2-deoxy-2-fluoro-1,3,5-tri-O-benzoyl-α-D-arabinofurance in the hydrobromic acid in acetic acid solution is carried out in a lower temperature environment with nitrogen gas. After completing the reaction, use organic solvents and alkaline solutions for washing. Then take the organic layer to dehydrate and concentrate by vacuum evaporation to get the product.

In the bromination, the weight percent of the hydrobromic acid in acetic acid solution used is ranging from 20% to 40% and 25% to 35% is preferred. The low temperature of the environment is ranging from −196° C. to 13° C. and an ice bath is preferred. The reaction time is ranging from 15 hours to 25 hours.

Using the above reactant and the reaction solution to react, the product obtained can be recovered by general solutions for elution. The recovery rate is up to 86% without complicated separation and purification steps. The method not only simplifies the steps effectively but also increases the yield rate and reduces waste of reactants significantly compared with the techniques available now.

Moreover, refer to the step S12, also perform another initial reaction to get a reactant required for preparing the precursor of the gene expression probe. Carry out silylation protection of a 5-bromouracil (5-BU) and a hexamethyldisilazane (HMDS) to get a 2,4-bis-O-(trimethylsilyl)-5-bromouracil, as shown in the following reaction formula two:

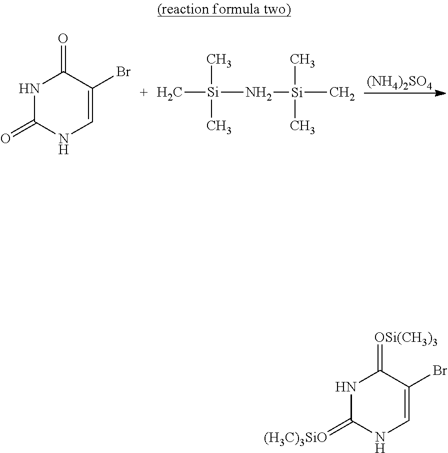

The silylation protection takes place in ammonium sulfate solution. Two trimethylsilyl groups of hexamethyldi-silazane are heated under a nitrogen gas flow to be connected to oxyl groups of 5-BU for silylation protection. Then remove the solvent to get a crude product and perform the following reactions without any separation and purification of the crude product.

The heating temperature is ranging from 120° C. to 160° C. and reaction time is from 2 hours to 6 hours.

Without separation and purification that cause yield loss, the product generated in the reaction of the step S12 has a high yield rate. The preparation cost required for the precursor of gene expression probe is lowered.

Refer to the step S14, use 2-deoxy-2-fluoro-3,5-di-O-benzoyl-α-D-arabinofuranosyl bromide produced in the step S11 and 2,4-bis-O-(trimethylsilyl)-5-bromouracil produced in the step S12 to perform a coupling reaction and get 1-(3,5-di-O-benzoyl-2-deoxy-2-fluoro-β-D-arabinofuranosyl)-5-bromouracil, represented by the following reaction formula three:

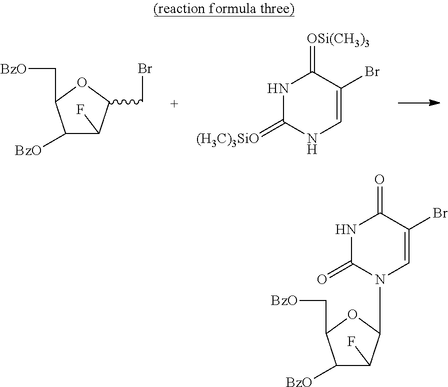

The 2-deoxy-2-fluoro-3,5-di-O-benzoyl-α-D-arabinofuranosyl bromide and 2,4-bis-O-(trimethylsilyl)-5-bromouracil are refluxed and stirred under a nitrogen gas flow. After completing the reaction, a solid product is obtained through vacuum concentration and washing with organic solvents.

As to the reflux mentioned above, the reaction temperature is ranging from 70° C. to 110° C. and the reaction time is from 15 hours to 25 hours.

In the coupling reaction, the reaction time is prolonged and the product is recovered by washing with only a bit organic solvent. Thus the complicated separation and purification steps of the techniques availble now are simplified. The purposes of higher yield rate and lower cost are also achieved. The yield rate of this step is still up to 64%, which is much more higher than other methods available now.

As shown in the step S16, use the 1-(3,5-di-O-benzoyl-2-deoxy-2-fluoro-β-D-arabinofuranosyl)-5-bromouracil generated in the step S14 and hexabutylditin to perform a substitution reaction in an anhydrous 1,4-dioxane solution and get 1-(3,5-di-O-benzoyl-2-deoxy-2-fluoro-β-D-arabinofuranosyl)-5-tributylstannyl)uracil, as shown in the following reaction formula four:

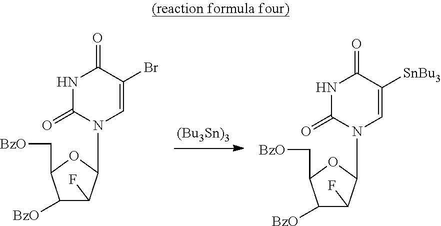

In the substitution reaction, the bromo group of the 1-(3,5-di-O-benzoyl-2-deoxy-2-fluoro-β-D-arabinofuranosyl)-5-bromouracil is oxidized and removed by catalyst. Then a tributylstannyl group of hexabutylditin replaces the bromo group on the six-member ring. The 1-(3,5-di-O-benzoyl-2-deoxy-2-fluoro-β-D-arabinofuranosyl)-5-bromouracil, hexabutylditin and catalyst are refluxed in an anhydrous 1,4-dioxane solution to obtain a crude product. Next the crude product is concentrated by vacuum evaporation and eluted by organic solvents. Then the organic solvents are separated and purified by liquid chromatography to get the 1-(3,5-di-O-benzoyl-2-deoxy-2-fluoro-β-D-arabinofuranosyl)-5-tributylstannyl)uracil.

The catalyst used in the substitution reaction can be palladium (Pd) catalyst, nickel (Ni) catalyst and copper (Cu) catalyst. The preferred catalyst is bis(triphenylphosphine)palladium chloride or tetrakis(triphenylphosphine)palladium(0).

As to the reflux of the substitution reaction, the reaction temperature is ranging from 90° C. to 130° C. and the reaction time is from 18 hours to 30 hours.

During the liquid chromatography, the organic solvent used to elute the crude product can be ether, ethyl acetate, and hexane. A column used in liquid chromatography is filled with materials having high affinity to the 1-(3,5-di-O-benzoyl-2-deoxy-2-fluoro-β-D-arabinofuranosyl)-5-tributylstannyl)uracil while silicon dioxide is preferred. An optimal mobile phase of the liquid chromatography is a mixed solution of ethyl acetate and hexane. However, the liquid chromatography conditions are not restricted.

By extending of the reflux time and using liquid chromatography for purification, the recovery/yield is effectively increased to 55%, 66% higher than that of the preparation method available now.

Refer to Step S18, carry out debenzoylation of 1-(3,5-di-O-benzoyl-2-deoxy-2-fluoro-β-D-arabinofuranosyl)-5-tributylstannyl)uracil to get 5-tributylstannyl-1-(2-deoxy-2-fluoro-β-D-arabinofuranosyl)uracil (FSAU), the target precursor of gene expression probe, as shown in the following reaction formula five:

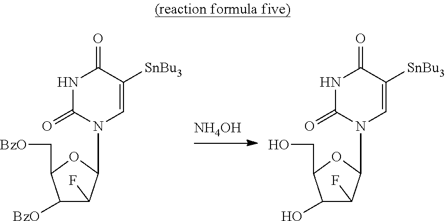

After 1-(3,5-di-O-benzoyl-2-deoxy-2-fluoro-β-D-arabinofuranosyl)-5-tributylstannyl)uracil being dissolved, add ammonium hydroxide solution into the solution and stir the mixture. After completing the reaction, the mixture is concentrated by vacuum evaporation and then separated and isolated by liquid chromatography.

The weight percent concentration of the ammonium hydroxide solution is ranging from 15% to 25%. The reaction temperature of the mixture being stirred is 20° C. to 30° C. and the reaction time is from 18 hours to 30 hours.

During the liquid chromatography, the column used is filled with materials having high affinity to the product FSAU while silicon dioxide is preferred. An optimal mobile phase of the liquid chromatography is a mixed solution of ethyl acetate and chloroform. Yet the liquid chromatography conditions are not restricted.

The above reaction process not only has easy steps but also gets intermediate products without using complicated separation and purification procedure. The liquid chromatography with better separation and purification effect is used for extraction of the final product. Thus the present invention indeed provides a method with higher yield/recovery rate and lower cost, representing a significant breakthrough in the related research.

After completing the preparation, the produced imaging agent precursor FSAU can be reacted with radioactive iodine and fialuridine labeled with radioactive iodine (I) is obtained by the substitution reaction. The fialuridine labeled with radioactive iodine (I) is used as a radioactive probe that reacts with a reporter gene for monitoring the expression of the therapeutic gene used in gene therapy.

In order to learn technical features, effects and implementation of the present invention, please refer to the following embodiments.

### Embodiment 1

### Synthesis of 2-deoxy-2-fluoro-3,5-di-O-benzoyl-α-D-arabinofuranosyl bromide

Add 0.9 g, 1.94 mmol starting material 2-deoxy-2-fluoro-1,3,5-tri-O-benzoyl-α-D-arabinofurance into 4 mL anhydrous dichloromethane and introduce nitrogen gas flow. Then drop a mixed solution of 1.25 mL 30 wt % hydrobromic acid in acetic acid solution and 5 mL anhydrous dichloromethane into the above solution of the starting material and stir the mixture for 20 hours.

Next add 20 mL dichloromethane into the mixture and then wash with 20 mL water and 20 mL sodium bicarbonate solution twice respectively. Take the organic layer of the product and dehydrate the organic layer by sodium sulfate solution. 2-deoxy-2-fluoro-3,5-di-O-benzoyl-α-D-arabinofuranosyl bromide is obtained after vacuum concentration.

Analytical data of 2-deoxy-2-fluoro-3,5-di-O-benzoyl-α-D-arabinofuranosyl bromide: IR (neat) ν 1726 (CO) cm−1. 1H NMR (CDCl3) δ 8.12-7.40 (m, 10H, ArH), 6.63 (d, 1H, H1), 5.68 (d, 1H, H2), 5.58 (d, 1H, H3), 4.85-4.70 (m, 3H, H4 and H5). 13C NMR (CDCl3) δ 165.99 and 165.51 (CO). 133.89, 133.23, 129.98, 129.78, 129.38, 128.64, 128.51 and 128.38 (Ph), 101.86 and 99.32 (CHF), 87.76 and 87.34 (CHBr), 84.70 (CHO), 77.43, 77.01, 76.58, 76.40 and 75.98 (CH2CH), 62.48 (CH2CH). MS m/z 343 (M+−Br).

### Embodiment 2

### Synthesis of 2,4-bis-O-(trimethylsilyl)-5-bromouracil

Take 0.32 g, 1.68 mmol 5-bromouracil (5-BU) as a starting material and add with 0.22 g, 1.68 mmol ammonium sulfate and 1.5 mL hexamethyldisilazane (HMDS) to form a mixture. Reflux and stir the mixture under nitrogen gas flow at 140° C. for 4 hours.

Then remove residual solvent by vacuum concentration to get a crude product. The crude product is directly used in the next step, without being separated and purified.

### Embodiment 3

### Synthesis of 1-(3,5-di-O-benzoyl-2-deoxy-2-fluoro-β-D-arabinofuranosyl)-5-bromouracil

Take 0.71 g, 1.68 mmol 2-deoxy-2-fluoro-3,5-di-O-benzoyl-α-D-arabinofuranosyl bromide and add 0.71 g, 1.68 mmol 2,4-bis-O-(trimethylsilyl)-5-bromouracil to be refluxed and stirred under nitrogen gas flow at 90° C. for at least 20 hours.

Then remove residual solvent by vacuum concentration, wash with hexane, filter by suction filtration, and wash again with a little ether to get the solid, 1-(3,5-di-O-benzoyl-2-deoxy-2-fluoro-β-D-arabinofuranosyl)-5-bromouracil.

Analytical data of 1-(3,5-di-O-benzoyl-2-deoxy-2-fluoro-β-D-arabinofuranosyl)-5-bromouracil: IR (KBr) ν1726, 1716 and 1671 (CO) cm−1. 1H NMR (CDCl3) δ 8.39 (s, 1H, NH), 8.11-8.03 (m, 4H, Ph), 7.92 (d, 1H, H6), 7.67-7.58 (m, 2H, Ph), 7.58-7.45 (m, 4H, Ph), 6.31 (dd, 1H, H1), 5.63 (dd, 1H, H3), 5.33 (dd, 1H, H2), 4.84-4.81 (m, 2H, H5), 4.56-4.52 (m, 1H, H4). 13C NMR (CDCl3) δ166.10 and 165.51 (CO). 158.10, 149.50, 141.30, 135.20, 133.80, 131.10, 129.80, 129.70 and 128.80 (Ph), 94.80 and 92.70 (CHF), 90.10 and 85.60 (CHBr), 82.10 (CHO), 78.10, 77.80, 77.60, 77.20 and 75.90 (CH2CH), 62.50 (CH2CH). MS m/z 534 (M)+.

### Embodiment 4

### Synthesis of 1-(3,5-di-O-benzoyl-2-deoxy-2-fluoro-β-D-arabinofuranosyl)-5-tributylstannyl)uracil

Take 0.51 g, 0.96 mmol 1-(3,5-di-O-benzoyl-2-deoxy-2-fluoro-β-D-arabinofuranosyl)-5-bromouracil and add 0.84 mL, 1.68 mmol hexabutylditin, 40 mg bis(triphenylphosphine)palladium chloride and 20 mL anhydrous 1,4-dioxane solution. Then the mixture is refluxed and stirred at 110° C. under nitrogen gas for 24 hours.

Next remove residual solvent by vacuum concentration, dissolve and collect precipitate by ether, ethyl acetate, and hexane, and remove insoluble materials. The above ether, ethyl acetate, and hexane and recovered material dissolved therein are separated and purified by liquid chromatography in which a silica gel column is used and a mixture of ethyl acetate and hexane (1:2) is as the mobile phase. Thus 1-(3,5-di-O-benzoyl-2-deoxy-2-fluoro-β-D-arabinofuranosyl)-5-tributylstannyl)uracil is obtained.

Analytical data of 1-(3,5-di-O-benzoyl-2-deoxy-2-fluoro-β-D-arabinofuranosyl)-5-tributylstannyl)uracil: IR (neat) ν 1715 and 1677 (CO) cm−1. 1H NMR (CDCl3) δ 8.97 (s, 1H, NH), 8.10-7.37 (m, 11H, Ph), 6.36 (dd, 1H, H1), 5.63 (dd, 1H, H3), 5.34 (dd, 1H, H2) 4.75 (m, 2H, H5), 4.51 (m, 1H, H4), 1.48-0.82 (m, 27H, SnBu3). 13C NMR (CDCl3) δ 165.98 and 165.15 (CO), 150.89, 143.93, 143.87, 134.07, 133.33, 129.95, 129.74, 129.33, 128.68, 128.45 and 128.16 (Ph), 112.13 (CHSn), 93.95 and 91.41 (CHF), 84.84 and 84.62 (CHCHO), 81.06 (CH2CH), 76.45 (CHN), 63.53 (CH2CH) 28.67, 27.10, 13.65, and 9.76 (SnCH2CH2CH2CH3)2. MS m/z 742 (M)+.

### Embodiment 5

### Synthesis of 5-tributylstannyl-1-(2-deoxy-2-fluoro-β-D-arabinofuranosyl)uracil (FSAU)

Dissolve 0.10 g, 0.13 mmol 1-(3,5-di-O-benzoyl-2-deoxy-2-fluoro-β-D-arabinofuranosyl)-5-tributylstannyl)-uracil in 4.5 mL methanol and add 4.5 mL 25% (v/v) ammonium hydroxide solution (NH₄OH) into the solution. Then the mixed solution is refluxed and stirred at 25° C. for 24 hours.

The product obtained is vacuum concentrated. Then the recovered material is separated and purified by liquid chromatography in which a silica gel column is used and a mixture of chloroform and ethyl acetate hexane (1:1) is as the mobile phase. Thus the final product, 5-tributylstannyl-1-(2-deoxy-2-fluoro-β-D-arabinofuranosyl)uracil (FSAU), is obtained.

Analytical data of 5-tributylstannyl-1-(2-deoxy-2-fluoro-β-D-arabinofuranosyl)uracil (FSAU): IR (neat) ν 3390 (OH), 2927 (NH), 1695 and 1647 (CO) cm−1. 1H NMR (CDCl3) δ9.62 (br, 1H, NH), 7.29 (d, 1H, CHSn), 6.25 (dd, 1H, H1), 5.09 (dd, 1H, H2), 4.46 (br, 1H, OH), 4.43 (dd, 1H, H3), 4.06 (q, 1H, H4), 3.85 (m, 2H, H5), 3.23 (br, 1H, OH), 1.54-0.85 (m, 27H, SnBu3). 13C NMR (CDCl3) δ 166.76 and 151.42 (CO), 144.91 (CHN), 111.99 (CHSn), 96.18 and 93.63 (C2), 84.35 and 84.15 (C3 and C4), 75.15 and 74.81 (C1), 61.92 (C5), 28.87, 27.22, 13.64 and 9.84 (SnCH2CH2CH2CH3)2. MS m/z 534 (M)+.

In summary, the method for preparing a precursor of gene expression probe of the present invention not only increases yield rate of the target product and accelerates production of the target product, but also improves production efficiency and reduces production cost. The acceleration of the production of the target product is achieved by simplifying the separation and purification steps available now. The yield rate of the target product is increased by reducing loss of the target product during production. The loss reduction measures include selection of wash solutions and reactive materials, modification of related steps, without over elution of intermediate products, and liquid chromatography for separating the final product. Moreover, the final product prepared by the present invention can be reacted with radioactive iodine to produce radioactive I¹²⁴-fialuridine that is able to freely pass through the cell membrane and react with the protein produced by the reporter gene used in the gene therapy. As an imaging probe used to monitor gene therapy, I¹²⁴-fialuridine has high commercial value.

In order to get or enhance the color, people skilled in the art can produce colored substrates according to the method of the present invention and followed by other treatments including painting, dyeing, etc. Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A method for preparing a precursor of gene expression probe comprising the steps of:
carrying out bromination of 2-deoxy-2-fluoro-1,3,5-tri-O-benzoyl-α-D-arabinofurance in a hydrobromic acid in acetic acid solution to get 2-deoxy-2-fluoro-3,5-di-O-benzoyl-α-D-arabinofuranosyl bromide;
also taking 5-bromouracil (5-BU) and hexamethyldisilazane (HMDS) to perform silylation protection and get 2,4-bis-O-(trimethylsilyl)-5-bromouracil;
performing a coupling reaction of 2-deoxy-2-fluoro-3,5-di-O-benzoyl-α-D-arabinofuranosyl bromide with 2,4-bis-O-(trimethylsilyl)-5-bromouracil to get 1-(3,5-di-O-benzoyl-2-deoxy-2-fluoro-β-D-arabinofuranosyl)-5-bromouracil;
performing a substitution reaction between 1-(3,5-di-O-benzoyl-2-deoxy-2-fluoro-β-D-arabinofuranosyl)-5-bromouracil and hexabutylditin in an anhydrous 1,4-dioxane solution to get 1-(3,5-di-O-benzoyl-2-deoxy-2-fluoro-β-D-arabinofuranosyl)-5-tributylstannyl)uracil; and
carrying out debenzoylation of 1-(3,5-di-O-benzoyl-2-deoxy-2-fluoro-β-D-arabinofuranosyl)-5-tributylstannyl)uracil to get 5-tributylstannyl-1-(2-deoxy-2-fluoro-β-D-arabinofuranosyl)uracil (FSAU).

2. The method as claimed in claim 1, wherein a weight percent of the hydrobromic acid in acetic acid solution used in the bromination step is from 20% to 40%.

3. The method as claimed in claim 1, wherein reaction temperature of the bromination step is from −196° C. to 13° C.

4. The method as claimed in claim 1, wherein reaction time of the bromination step is from 15 hours to 25 hours.

5. The method as claimed in claim 1, wherein an ammonium sulfate solution and hexamethyldisilazane are used in the silylation protection step.

6. The method as claimed in claim 1, wherein reaction temperature of the silylation protection step is from 120° C. to 160° C.

7. The method as claimed in claim 1, wherein reaction time of the silylation protection step is from 2 hours to 6 hours.

8. The method as claimed in claim 1, wherein reaction temperature of the coupling reaction step is from 70° C. to 110° C.

9. The method as claimed in claim 1, wherein reaction time of the coupling reaction step is from 15 hours to 25 hours.

10. The method as claimed in claim 1, wherein a catalyst used in the substitution reaction step is bis(triphenylphosphine)palladium chloride.

11. The method as claimed in claim 1, wherein reaction temperature of the substitution reaction step is from 90° C. to 130° C.

12. The method as claimed in claim 1, wherein reaction time of the substitution reaction step is from 18 hours to 30 hours.

13. The method as claimed in claim 1, wherein the debenzoylation takes place in an ammonium hydroxide solution whose weight percent concentration is from 15% to 25%.

14. The method as claimed in claim 1, wherein reaction temperature of the debenzoylation step is from 20° C. to 30° C.

15. The method as claimed in claim 1, wherein reaction time of the debenzoylation step is from 18 hours to 30 hours.

16. The method as claimed in claim 1, wherein a substitution reaction of 5-tributylstannyl-1-(2-deoxy-2-fluoro-β-D-arabinofuranosyl)uracil (FSAU) with radioactive iodine is performed to get fialuridine labeled with radioactive iodine (I) after the debenzoylation.
